(19) United States
(12) Patent Application Publication
(10) Pub. No.: US 2006/0121058 A1
(43) Pub. Date: Jun. 8, 2006

(54) Anti-pneumococcal preparations

(75) Inventors: Richard Malley, Beverly, MA (US); Porter Anderson, Fripp Island, SC (US)

Correspondence Address: David S. Resnick;NIXON PEABODY LLP, 100 Summer Street, Boston, MA, 02110, US

(73) Assignees: Children's Medical Center Corporation, Boston, MA (US); University of Rochester Office of Technology Transfer, Rochester, NY (US)

(21) Appl. No.: 11/268,694

(22) Filed: Nov. 7, 2005

Related U.S. Application Data
(63) Continuation of application No. 10/637,896, filed on Aug. 8, 2003.
(60) Provisional application No. 60/401,896, filed on Aug. 8, 2002.

(51) Int. Cl. A61K 39/09; A61K 31/739

(52) U.S. Cl. 424/244.1; 514/54

(57) ABSTRACT

Disclosed are compositions and methods for treating, reducing or preventing diseases and infections caused by pneumococci. The methods and compositions rely on the use of a CPS-containing composition (e.g., a CPS teichoic acid polymer preparation) of S. pneumoniae and an adjuvant, which may be chemically conjugated or simply admixed. The methods and compositions are particularly useful against nasopharyngeal colonization and invasive disease due to encapsulated pneumococci.

This application claims the benefit of U.S. provisional application No. 60/401,896, which was filed on Aug. 8, 2002. The contents of the prior application are hereby incorporated by reference in their entirety.

### TECHNICAL FIELD

The present invention relates to compositions and methods for preventing illnesses associated with pneumococcal infection. The compositions contain an antigen such as teichoic acid polymer of Streptococcus pneumoniae (or a variant or derivative thereof, as described below), and they can be formulated for nasopharyngeal administration.

### BACKGROUND

Streptococcus pneumoniae (S. pneumoniae or pneumococcus) is a pathogenic, gram-positive bacterium that can be classified as having one of about 100 antigenic serotypes, depending on the capsular polysaccharide expressed external to the bacterial cell wall. S. pneumoniae causes a variety of undesirable conditions, including pneumonia, middle ear infections (otitis media), bacteremia, and bacterial meningitis, and it can exacerbate other conditions, such as chronic bronchitis, sinusitis, arthritis, and conjunctivitis.

Compositions that contain a teichoic acid polymer present in pneumococci of all capsular serotypes has been evaluated as a protective antigen, and compositions including preparations of the polymer or its components have been tested as a possible vaccine in animal models (Briles et al., J. Exp. Med. 153:694-705, 1981; Wallick et al., J. Immunol. 130:2871-2875, 1983; Szu et al., Infection and Immunity 54:448455, 1986; Sorenson et al., Infection and Immunity 56:1890-1896, 1988; Musher et al., J. Infect. Dis. 161:736-740, 1990; Nielsen et al., Microb. Pathogen. 14:299-305, 1993; Briles et al., Clinical Microbiology Reviews 11:645-657, 1998).

Pneumococci can contain several morphologic forms of the polymer. One is associated with the cell wall and has been called cell polysaccharide or CPS. Preparations of CPS may thus contain residual fragments of the cell wall peptidoglycan. However, certain strains of pneumococci were experimentally generated in which the original serotype capsular polysaccharide was deleted and replaced with a capsule-like external layer consisting of the teichoic acid polymer, hyperproduced. Commercially available “CPS” preparations are typically made from such strains, although the morphologic origin is the capsule and not the cell wall. In yet another form, originally called Forssman or F antigen, the teichoic acid has a glycolipid end group that causes the polymer to associate with the (phospholipid) cell membrane of the pneumococcus; this form has more recently been called lipoteichoic acid or LTA. The teichoic acid polymer (FIG. 1) of these several forms is identical (Fisher, “Pneumococcal Lipoteichoic Acid and Teichoic Acid” In Streptococcus pneumoniae—Molecular Biology and Mechanisms of Disease, pp. 155-177, A. Tomasz, Ed., Mary Ann Liebert, Inc., Larchmont N.Y., 2000). Typical preparations can contain polymer without and with the peptidoglycan fragments or the glycolipid group. For brevity, we refer to any such preparations below as CPS.

Serum antibodies to phosphoryl choline, a component of CPS, may protect mice against parenteral challenge with capsulated serotype 3 pneumococci (Briles et al., supra). In addition, compositions including phosphoryl choline conjugated to a carrier protein and mixed with Freund's adjuvant may protect mice against an intravenous challenge with serotype 1 or serotype 3 pneumococci (Wallick et al., supra). Moreover, serum transferred from treated mice passively protected untreated mice (Wallick et al., supra). However, in a similar study, parenterally injected CPS that was coupled to a protein carrier failed to protect mice against challenges with type 3 or 6A pneumocci, and antibodies that were raised in rabbits with that composition (i.e., CPS coupled to a protein carrier and mixed with Freund's adjuvant) failed to passively protect mice against similar challenges (Szu et al., supra). Other investigators also concluded that serum antibodies to CPS, whether directed against phosphoryl choline or another component of CPS, fail to provide protection against capsulated pneumococci (Sorensen et al., Musher et al., and Nielsen et al., supra). Given these findings, interest in CPS as an immunizing agent for pneumococcal infection has all but disappeared.

While the compositions described above have been tested in animals, a few pneumococcal vaccines based upon the capsular polysaccharides are available for use in humans. The vaccines intended for immunologically mature humans include a combination of unconjugated pneumococcal polysaccharides. These are PNEUMOVAX® 23 (Merck Sharp & Dohme, West Point, Pa.), which includes 23 different purified pneumococcal polysaccharides, and PNU-IMMUNE® 23, which is a similar vaccine produced by Wyeth-Lederle Vaccines (Pearl River, N.Y.). These vaccines represent up to 90% of the serotypes that cause invasive pneumococcal infections in the United States, including the six serotypes that most frequently cause invasive drug-resistant pneumococcal infection. A vaccine specifically designed for infants is PREVNAR®, which consists of seven serotypes of pneumococcal polysaccharides (4, 6B, 9V, 14, 18C, 19F, and 23F according to the Danish nomenclature) that have been conjugated to the protein CRM-197 (also produced by Wyeth-Lederle Vaccines).

Unfortunately, vaccines based upon capsular polysaccharides are not ideal. For example, unconjugated pneumococcal polysaccharide vaccines are not very effective in young children (Douglas et al., J. Infect. Dis. 148:131-137; Ahonkai et al, New Engl. J. Med. 301:26-27, 1979; and Sell et al., Rev. Infect. Dis. 3:S97-S107, 1981). While the conjugated vaccine is effective in infancy, the number of serotypes that can be included in those vaccines is limited; serotype replacement may occur (serotype replacement has been reported in clinical trials of PREVNAR® for otitis media); and production is costly. Thus, there is a need for simpler, more economical compositions (and methods of administering those compositions) that confer better protection on subjects of all ages against more serotypes, and preferably all serotypes, of S. pneumoniae.

### SUMMARY

The present invention provides compositions that contain the CPS of S. pneumoniae and/or one or more portions, fragments, or derivatives thereof that retain a sufficient degree of activity (e.g., immunogenicity) to function in the methods described herein. The term “CPS-containing composition” is used herein to describe compositions containing the pneumococcal teichoic acid polymer in any of the variant forms described herein (e.g., a teichoic acid-containing composition can contain all or part of the polymeric backbone, and/or phosphoryl choline groups that extend therefrom, and/or glycolipid endgroups, and/or other appended components such as amino acids or other components of the cell wall peptidoglycan ; the term CPS is also used to refer to any form of a polymer whose published repeating structure is shown in FIG. 1, regardless of its morphologic origin in the pneumococcus, or to slight variants of this structure based upon the number of phosphoryl choline groups. CPS-containing compositions are further described below.

The CPS-containing compositions of the invention, following administration to a patient, can reduce the likelihood that the patient will develop a disease associated with S. pneumoniae. While patients amenable to treatment are described further below, we note here that the compositions of the invention are most likely to be administered to inhibit bacterial colonization and reduce the ability of the bacteria to gain access to a part of the patient's body that should be bacteria-free (e.g., an organ or tissue other than the throat). Many patients carry pneumococci in their throat; it is only when the bacteria spread beyond that non-sterile region that disease occurs. Accordingly, and preferably, the CPS-containing compositions described herein are administered to patients who, while possibly carrying pneumococci, appear to be healthy. Thus, the invention features methods of administering a CPS-containing composition to a patient who may develop a disease associated with a pneumococcal infection (the term “disease” is meant to encompass any illness, whether that illness is typically referred to as a disease, syndrome, condition, or the like). For example, the patient can develop otitis media or pneumonia, or a disease that occurs when infection spreads to other organs or organ systems (e.g., bacterial meningitis or bacteremia) (the patient population is described further below).

In addition, a CPS-containing composition can contain an adjuvant. For example, in addition to containing CPS and/or one or more portions, fragments, or derivatives thereof (including portions, fragments, or derivatives where the CPS polymer carries a lipid or glycolipid (e.g., CPS in which teichoic acid is covalently linked to a portion of CPS (e.g., to muramic acid))), the compositions of the invention can include an adjuvant and they can be formulated for mucosal administration (i.e., formulated for administration in a way that brings them into direct contact with any mucosal surface, such as a nasopharyngeal, oropharyngeal, or pharyngeal surface). Other routes of administration include oral (in which the patient may, e.g., swallow a CPS-containing composition) enteral (in which a CPS-containing composition is otherwise administered to the digestive tract), or pulmonary (in which the CPS-containing composition is administered to the respiratory tract at some point that includes application distal to the nasopharynx). Accordingly, CPS-containing compositions can be prepared for administration through a nebulizer or similar device or as a topical solution (e.g., a wash, lotion, gel, salve, or the like). The compositions of the invention can also be prepared as freeze-dried preparations. An adjuvant suitable for mucosal (e.g., nasal) administration can be included, regardless of the precise configuration of the CPS (e.g., regardless of whether a fragment or portion of a naturally occurring CPS is used as the antigen, regardless of the strain(s) of S. pneumonaie from which the CPS is derived, and regardless of whether or not the CPS is conjugated to another agent such as a lipid or glycolipid) and the compositions can be administered mucosally (e.g., by way of the patient's nose or the mucosa in or around the mouth and throat). Thus, as noted above, the compositions of the invention can be used to protect a patient from (e.g., reduce the risk or extent of) pneumococcal colonization, which can occur via the nasopharynx and which is preliminary to pneumococcal disease.

Although the traditional adjuvants developed for parenteral vaccination are not suitable, a variety of adjuvants can be included in the CPS-containing compositions of the invention. For example, one can use the bacterial protein toxins known to facilitate antigen presentation across mucosal surfaces. For example, one can use cholera toxin (CT), Escherichia coli heat-labile toxin (LT), pertussis toxin, shiga toxin, anthrax toxin, pseudomonas exotoxin A, or nontoxic derivatives of such toxins. Derivatives can be rendered nontoxic by, for example, mutating the nucleic acid sequence that encodes them; some amino acid substitutions are known to reduce toxicity while retaining adjuvant action (e.g., LT(R102G) or LT(S63K)). Alternatively, one can use only the subunits of the toxins responsible for cell binding (e.g., “B subunits” such as CT-B and LT-B). See Mrsny et al., Drug Discovery Today 7:247-258, 2002; see also Holmgren et al., Vaccine 11:1179-1184, 1993; the entire contents of these references is hereby incorporated by reference). Protein adjuvants of this kind can simply be combined or admixed with the antigens to be mucosally administered. Alternatively, such adjuvants can be conjugated to (a broad term that encompasses any coupling or association) the antigen (i.e., to CPS or a portion, fragment, or derivative thereof). For example, CPS and a protein adjuvant can be chemically coupled by the technique described by Szu et al. (supra). In addition to the proteinaceous adjuvants listed above, one can use a non-proteinaceious adjuvant (e.g., the adjuvant can be a liposome or a lipid-based composition; the adjuvant can be Rhinovax; see below). Moreover, in addition to (or as an alternative to) conventional adjuvants, the CPS-containing compositions of the invention can include heterologous carrier proteins that are chemically coupled to the antigen and that enhance the immunogenicity of the CPS-containing compositions, particularly in immunologically immature subjects. For example, a carrier protein can be conjugated to CPS (or a portion, fragment, or derivative thereof) and administered with (e.g., admixed with) one of the adjuvants described herein.

Regardless of the precise content, CPS-containing compositions of the invention will be physiologically or pharmaceutically acceptable compositions. The carriers can be (or can include), for example, water or saline (e.g., sterile water or sterile, isotonic saline), dextrose, glycerol, ethanol, or a combination thereof.

In other embodiments, the compositions of the invention can include antibodies (monoclonal or polyclonal antibodies) generated against S. pneumoniae CPS including the variants described herein. These compositions can also be formulated for mucosal administration, and they can be administered to patients by that route (e.g., anti-CPS antibodies can be administered to the nasal or oral mucosa). These antibody-containing compositions are believed to confer passive immunity against infection by (or disease associated with) S. pneumoniae.

### BRIEF DESCRIPTION OF THE DRAWING

FIG. 1 is a published representation of the teichoic acid polymeric structure of pneumococcal CPS (reproduced from Szu et al., Infection and Immunity 54:448-455, 1986). Variants based upon the number of phosphoryl choline groups have recently been reported (Karlsson et al, Eur. J. Biochem. 275:1091) and are within the scope of the invention.

### DETAILED DESCRIPTION

The compositions and methods described herein are based, in part, on the discovery that a CPS-containing preparation of S. pneumoniae can be formulated for mucosal (e.g., intranasal) administration and, when administered to a subject by that route, can reduce the risk of disease associated with S. pneumoniae. As discussed further below, administration can begin before a subject has a pneumococcal disease (i.e., the preparation can be administered prophylactically), after suspected exposure to a pneumococcal disease (but while the patient is still apparently healthy), or after a subject has apparently recovered from a pneumococcal disease (i.e., the preparation can be administered to reduce the likelihood of recurrent infection or disease). In alternative embodiments, the CPS-containing compositions can include, or can be administered with, an adjuvant (e.g., an adjuvant suitable for the type of patient being treated (e.g., human patients) and known to increase immunogenicity by mucosal routes). In other embodiments, the antigens can be conjugated or conjugated to a carrier and, optionally, administered with an adjuvant.

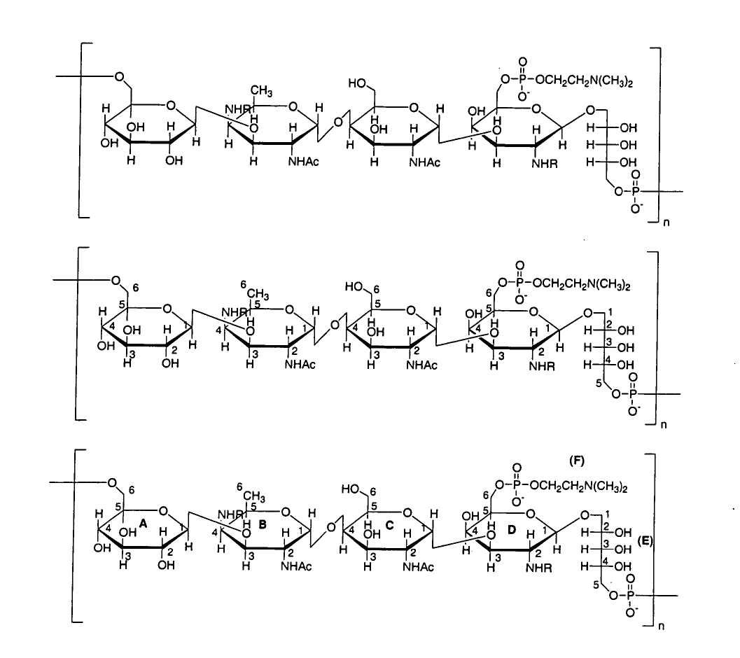

Antigens: Pneumococci can contain several morphologic forms of the polymer teichoic acid, a published structure of which is shown in FIG. 1. One form of teichoic acid is associated with the cell wall, and that form has been called cell wall polysaccharide (or CPS). Preparations of CPS, including those useful in the physiologically acceptable compositions of the present invention, may thus contain residual fragments of the cell wall peptidoglycan. In certain strains of experimentally generated pneumococci, the original serotype capsular polysaccharide was deleted and replaced with a capsule-like external layer including the teichoic acid polymer; some commercial sources of “cell wall polysaccharide” are in fact isolated from such capsular layers. In yet another morphological form, originally called Forssman or F antigen, the teichoic acid moiety includes a glycolipid end group that facilitates interaction between the polymer and the phospholipid cell membrane of pneumococcus. This form of CPS has more recently been called lipoteichoic acid or LTA. The teichoic acid polymer of these several forms is essentially identical. Further, teichoic acid that is substantially identical to any of the forms described here can be a part of the CPS-containing compositions of the present invention. For example, typical preparations can contain teichoic acid polymers with or without the peptidoglycan fragments or the glycolipid groups.

The compositions of the invention include CPS or a portion, fragment, or derivative thereof (such as described above) that is immunologically active. CPS (or a portion, fragment, or derivative thereof) is “immunologically active” when, upon administration (e.g., mucosal administration) to a mammal, it evokes an immune response (either humoral or cellular) in the mammal (all that is required is an immune response sufficient to benefit the patient). The portion or fragment of CPS may be all or a portion of the CPS backbone (see, e.g., FIG. 1) or all or a portion of the phosphoryl choline moiety that extends from the backbone (see, e.g., FIG. 1).

CPS can be obtained from a commercial source (e.g., Statens Seruminstitut, Copenhagen, Denmark) (some adjuvants, such as toxin-derived adjuvants are also commercially available)), or it may be extracted or purified from, for example, bacterial (S. pneumoniae) cells. Strains of bacteria (e.g., S. pneumoniae and those that produce native toxins as well as detoxified derivatives) can be obtained from the American Type Culture Collection (ATCC; Manassas, Va.). Strains of S. pneumoniae that hyperproduce CPS as a capsule-like structure can also be obtained from the ATCC, and CPS can be purified by published techniques. For example, CPS can be isolated from culture supernatants as well as from bacterial cells from the base extraction technique described in U.S. Pat. No. 6,248,570 (the contents of which is incorporated herein by reference). The CPS produced by that method may lack covalent attachment to extraneous peptidoglycan. CPS-containing compositions that include LTA can be extracted from pneumococci by a chloroform-methanol procedure and purified by hydrophobic affinity chromatography (Fischer, Pneumococcal Lipoteichoic Acid and Teichoic Acid In: Streptococcus pneumoniae—Molecular Biology and Mechanisms of Disease, pp. 155-177, A. Tomasz, Ed., Mary Ann Liebert, Inc., Larchmont, N.Y., 2000, which is hereby incorporated by reference in its entirety).

Other purification methods rely on treating the source cells with mutanolysin, which cleaves the bacterial wall and frees cellular components. The cell lysates can be treated with additional enzymes to remove proteins and nucleic acids, and purification can be carried out by differential precipitation and chromatography (Wessels et al., Infect. Immunol. 57:1089-1094, 1989; Wessels et al., J. Clin Invest. 86:428-433, 1990).

Conjugation: As noted above, antigenic components can be conjugated (i.e., linked by covalent bonds) to other molecules to increase immunogenicity. The antigenic components can also be conjugated to lipid or glycolipid molecules or to protein adjuvants or carrier proteins by methods known in the art (“proteins” and “peptides” are both polymers of amino acid residues and either may be used in the present invention; the term protein is used here only because it is more commonly applied to the higher molecular weight polymers used as adjuvants and carriers). CPS or a portion, fragment, or derivative thereof that is immunologically active can be thus conjugated by methods that take account of the particular structure of CPS which, like most proteins, contains free amino groups and which contains an easily hydrolyzed phosphodiester linkage (Fischer et al., Eur. J. Biochem. 215:851-857, 1993). Amino acids amenable to conjugation may also extend from a teichoic acid backbone.

One suitable conjugation procedure is described in Szu et al. (supra.), in which the reagent N-succinimidyl-3-(2-pyridyldithio)-proprinate (SPDP) is first coupled separately to the free amino groups of the CPS and the protein, and the two adducts are coupled by disulfide bond exchange. In another suitable method, CPS is selectively cleaved by periodate oxidation giving immunogenic fragments with aldehyde termini, the free amino groups of the fragments are reversibly blocked with the reagent “t-BOC”, the aldehyde groups are coupled to the free amino acid groups of the protein by reductive amination (Anderson et al., J. Immunol. 137:1181-1186, 1987), and the blocking groups are then removed. While specific conjugation methods are described herein, the invention is not so limited. Any suitable mode of conjugation may be employed to conjugate the CPS component with an adjuvant or carrier.

CPS-containing conjugates include conjugates in which a protein or peptide is linked to the CPS through one or more sites on the CPS. Accordingly, the CPS-containing compositions can include conjugate molecules that are monomers, dimers, trimers and/or more highly cross-linked molecules, the CPS cross-linking multiple proteins.

Adjuvants: Adjuvants enhance the immunogenicity of an antigen but are not necessarily immunogenic themselves. In the context of the present invention, particularly where CPS-based compositions are administered to the mucosa, it is desirable to use an adjuvant that facilitates antigen presentation across mucosal surfaces for delivery to antigen-processing cells below. Certain bacterial protein toxins and their derivatives or sub-components have this capacity. For example, one can use cholera toxin (CT), Escherichia coli heat-labile toxin (LT), pertussis toxin, shiga toxin, anthrax toxin, pseudomonas exotoxin A, or nontoxic derivatives of such toxins. See Mrsny et al., Drug Discovery Today 7:247-258, 2002. Pneumolysin, a toxin from the pneumococcus itself, may likewise act as a mucosal adjuvant. Derivatives can be rendered nontoxic by, for example, mutating the nucleic acid sequence that encodes them; some amino acid substitutions are known to reduce toxicity while retaining adjuvant action (e.g., LT(R102G) or LT(S63K)). Alternatively, one can use only the inherently nontoxic subunits of the toxins responsible for cell binding (e.g., “B subunits” such as CT-B and LT-B). See Holmgren et al., Vaccine 11:1179-1184, 1993). Such adjuvants can simply be combined or admixed with the antigens to be mucosally administered. Alternatively, protein adjuvants can be conjugated to (a broad term that encompasses any coupling or association) the antigen (i.e., to CPS or a portion, fragment, or derivative thereof). For example, CPS and a protein adjuvant can be chemically coupled as described by Szu et al. (supra). In addition to the proteinaceous adjuvants listed above, one can use a non-proteinaceious adjuvant (e.g., the adjuvant can be a liposome, a lipid-based composition such as Rhinovax, or a glycolipid such as derivitives of the lipid A component of Gram-negative bacterial endotoxin).

Ideally, the adjuvant selected will: (1) lack toxicity; (2) stimulate a long-lasting immune response; (3) remain stable despite long-term storage; (4) elicit humoral and perhaps cellular responses to CPS; (5) act synergistically with other adjuvants; (6) selectively interact with populations of antigen presenting cells (APC); specifically elicit appropriate THI or TH 2 cell-specific immune responses; and (8) electively increase appropriate antibody isotype levels (for example IgA) against antigens. Of course, adjuvants having fewer than all of these characteristics can still be used.

Carrier Proteins: Moreover, in addition to (or as an alternative to) such adjuvants, the CPS-containing compositions of the invention can include heterologous carrier proteins that are chemically coupled to the antigen. These include proteins that may lack adjuvant activity when admixed but that, when coupled, enhance the presentation of the CPS-containing compositions to antigen-processing cells, particularly useful in immunologically immature subjects. For example, CPS (or a portion, fragment, or derivative thereof) can be conjugated to the outer membrane protein complex of a Gram-negative bacterium such as Neisseria meningitidis (or proteins within those complexes), to various bacterial toxins and toxoids (e.g., diphtheria or tetanus toxins or their respective conventional toxoids or genetically detoxified toxins referred to as cross-reacting material, e.g., CRM 197), or haemocyanins (some of these materials are mentioned elsewhere herein).

The effect of the linkage to carriers may be additive to that of the above-described adjuvants. For example, a carrier protein can be conjugated to CPS and administered with (e.g., admixed with) one of the adjuvants described herein.

Formulations: When a CPS-based composition is formulated for intranasal delivery, it can be formulated as a spray or the like (e.g., a nasal spray, aerosol spray, or pump spray). Aerosol spray preparations can be contained in a pressurized container with a suitable propellant such as a hydrocarbon propellant. Pump spray dispensers can dispense a metered dose or a dose having a specific particle or droplet size. Any dispensing device can be arranged to dispense only a single dose, or a multiplicity of doses. More generally, compositions of the invention, especially those formulated for intranasal administration, can also be provided as solutions, suspensions, or viscous compositions (e.g., gels, lotions, creams, or ointments).

The CPS-containing compositions of the invention (we reiterate here that “CPS-containing” encompasses CPS as well as portions, fragments, and derivatives thereof) can also include “auxiliary” substances, such as wetting agents, emulsifying agents, dispersing agents, thickening agents, or pH buffering agents. When formulated for mucosal administration, the compositions of the invention, with or without auxiliary substances, can include a substance to inhibit drying of the mucosal membrane and one or more substances to prevent irritation of the mucosal membrane. In addition, any of the compositions of the invention can contain preservatives such as benzyl alcohol, chlorobutanol, and parabens.

CPS-based compositions can also be formulated as powders by methods known in the art (e.g., by freeze-drying). When so formulated, the composition can be administered by inhalation. Alternatively, the powder formulation can be resuspended prior to use.

The dose of CPS (or any portion, fragment, or derivative thereof) will be a dose that is safe and effective (i.e., able to generate a protective immune response in a patient upon the completion of the treatment protocol) and appropriate for a particular type of formulation. Suitable dosages of antigens are determined by those of ordinary skill in the art by routine methods, and the dosage can range from micrograms to milligrams. For example, an average adult human can be given 1 μg-10 mg of CPS (or a portion, fragment, or derivative thereof) (e.g., 1 μg-1 mg; 1 μg-200 μg (e.g., 10 or 20 μg-100 to 200 μg); or 10 μg-50 μg (e.g., 10, 15, 20, 25, 30, 35, 40, 45, or 50 μg)). These doses can be administered on one or several (e.g., two, three, or four successive occasions) and can be varied appropriated based on the patient's age, body weight and other known criteria used to determine dosages of various drugs and antigens. The antibody response in an individual can be monitored by assaying for antibody titer or bactericidal activity and boosted if necessary to enhance the response.

Routes and Methods of Administration: The CPS-containing compositions of the present invention can be administered to any mucus membrane or mucosal surface, and (as described above) compositions can be formulated for that route of administration and can include adjuvants that are effective when so administered. More specifically, the CPS-containing compositions of the present invention can be administered to the nasal mucosa, the oral cavity, the throat, or the lungs. Without limiting the invention to compositions that exert a beneficial effect by any particular mechanism, we believe that compositions administered to one or more of these regions will inhibit bacterial colonization and subsequent systemic invasion (or invasion to organs such as the ear or brain). Compositions containing anti-CPS antibodies can be similarly formulated and administered. Here again, the compositions may produce a localized benefit (antigen administered parenterally produces serum antibodies that may have limited access to the mucosa).

Patients and Conditions Amenable to Treatment: The patient can be any animal susceptible to pneumococcal infection. For example, the patient can be a mammal, such as a human, a domesticated animal (e.g., a dog or cat; or a farm animal such as a horse, pig, or cow), or a rodent. Unless specifically noted, the “patient” may also be referred to herein as a “host” or “subject.”

The compositions of the invention can be administered to patients of any age, including patients whose immune systems are not fully mature (e.g., children under the age of two). In that event, conjugated antigens may be administered at, for example, about two months of age, and conjugated or unconjugated antigens can be administered subsequently (at, for example, about four, six, 12, and/or 18 months of age. CPS-based compositions can also be administered to elderly patients (e.g., patients over the age of 65). While otherwise healthy patients may also be treated, the ability to mount an immune response against the immunogen need not be perfect for the CPS-based compositions described here to confer some degree of protection against S. pneumoniae. Physicians, veterinarians, and others who routinely care for patients will be able to determine whether a patient is apparently in good enough health to consider administering a CPS-based composition.

The CPS-based compositions of the present invention are also useful as components of multivalent vaccines, which are capable of eliciting an immune response against a plurality of infectious agents.

The invention is illustrated further by the following non-limiting examples, which demonstrate that intranasal administration of CPS, with cholera toxin (CT) as an adjuvant, exhibits a significant and impressive dose-dependent protection against intranasal S. pneumoniae infection.

### EXAMPLES

### Example 1

### Protection of Mice Against Experimental Nasopharyngeal Colonization with Serotype 6B Pneumococci using the Combination of CPS and an Adjuvant

An experiment was conducted to evaluate protection conferred by a vaccine composition comprising CPS and an adjuvant, as compared to the possible nonspecific effect of adjuvant alone, against pneumococcal infections in mice. C57BL/6J mice at 4-6 weeks of age were divided into two experimental groups: one group of mice were immunized intranasally with 500 micrograms of CPS (pneumococcal cell wall polysaccharide (C-Ps), Article 3459, Lot E6, from Statens Seruminstitut, Copenhagen, Denmark) mixed with 1 microgram of the adjuvant cholera toxin (CT), while the control group was immunized with CT only. These immunizations were done at week 0 and week 1 of the experiment. Three weeks after the second immunization, the mice were infected with pneumococcus type 6B strain GA03212, 10⁶ cfu, in 10 μl of saline intranasally. One week after the exposure to the live capsulated pneumococci, the mice were examined for the number of viable pneumococci in the upper respiratory tract as described in Malley et al. (Infection and Immunity 69:4870-4873, 2001).

The experimental results are summarized below.

| Immunogen | Mice colonized/total challenged |
|---|---|
| Cholera toxin alone, 1 microgram (CT) | 12/16* |
| 500 micrograms CPS + CT | 4/15* |

*p = 0.012

These results demonstrate significant protection by the combination of CPS and cholera toxin. In addition, when the density of pneumococcal colonization between immunized animals and control animals was compared, control animals had a significantly higher density of colonization than CPS immunized animals (p<0.001).

### Example 2

This experiment was conducted to test the necessity for adjuvant, to examine the effect of the dosage of CPS when given by the intranasal (i.n.) route, and to compare the effect to that of CPS given by a parenteral route. The experiment was carried out as described in Example 1 with the following modifications: the mice were divided into six experimental groups in which the immunizations varied as follows: (a) 1 microgram (μg) of cholera toxin (CT), given i.n.; (b) 200 μg of CPS alone, given i.n.; (c) 200 μg of CPS with CT, given i.n.; (d) 20 μg of CPS with CT, given i.n.; (e) 2 μg of CPS with CT, given i.n.; or (f) 5 μg of CPS alone, injected intraperitoneally (i.p.), a commonly used parenteral route for mice. This immunization was expected from previous experience to generate a moderate serum antibody response similar to that of immunization c. above.

The results, shown in Tables 1 and 2 (below), support the following conclusions.

1. Neither the CPS nor the CT adjuvant alone is substantially protective by the intranasal route; the combination produces a better result (compare groups a, b, and c).

2. The protective effect of CPS (with CT) increases with dosage, becoming significant between 20 and 200 μg (compare groups a, c, d, and e).

3. The mice immunized by the parenteral route (group f) were not protected, a result consistent with the above-cited experience of other workers such as Szu et al. The interpretation of this result requires consideration of the titers of serum antibodies against CPS determined just before challenge. The geometric mean titers of CPS antibodies as determined by enzyme-linked immunoassay are given in Table 2 for mice of groups a, c, and f.

Compared to control group a., both the intranasally immunized (and protected) group c. and parenterally immunized (not protected) group f. had similar increases (4-5 fold) in antibodies. Thus the protective effect of mucosally applied CPS with CT adjuvant may come from the particular qualities of the immune response (such as secretory antibody) rather than the overall serum antibody response.

TABLE 1

| | | # of mice colonized/ |
|---|---|---|
| Group Immunogen (Route of administration) | | total # mice challenged |
| a. Cholera toxin, 1 μg (CT) | i.n. | 11/16 |
| b. CPS (200 μg) | i.n. | 13/15 |
| c. CPS (200 μg + CT) | i.n. | 4/12 |
| d. CPS (20 μg + CT) | i.n. | 10/16 |
| e. CPS (2 μg + CT) | i.n. | 12/16 |
| f. CPS (5 μg) | i.p | 11/12 |

TABLE 2

| Group Immunogen (Route of administration) | | Titer of CPS antibodies |
|---|---|---|
| a. CT | i.n | 206 |
| c. CPS, 200 μg + CT | i.n. | 1075 |
| f. CPS, 5 ug | i.p. | 920 |

### Example 3

The following study was carried out to test for protection against middle ear infection as well as nasopharyngeal colonization of mice. The experiment was conducted similar to the study described in Example 1, except that, in addition to quantitative culture of the upper respiratory tract, the middle ear exudates was cultured for pneumococci and examined by microscopy with gram stain.

| | Geometric mean of | | | | | |
|---|---|---|---|---|---|---|
| | pneumococci/ml | | | | | |
| Vaccine or | in wash of upper | | | Mice colonized in the | | |
| control | respiratory tract | | | middle ear/total | | |
| CT | 1400 | | | 11/12 | | |
| | | {close oversize brace} | p = 0.05 | | {close oversize brace} | p = 0.018 |
| CT + 200 ug CPS | 120 | | | 3/8 | | |

In all but one of the CT control animals, the middle ear as well as the upper respiratory tract was colonized. In those receiving the adjuvant plus CPS, in addition to reduced colonization of the upper tract, there was significant reduction of culture-positive middle ear exudates. In the middle ear exudates with pneumocci, numerous polymorphonuclear leukocytes were seen in the gram stains, but were not present in the sterile samples from culture-negative mice. Thus, the presence of the pneumococci appeared to represent infection rather than benign colonization of the middle ear. Therefore, the combination of CPS and an appropriate adjuvant have a potential for prevention of otitis media.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method for inhibiting S. pneumoniae bacterial colonization and reducing the ability of: the Streptococcus pneumoniae bacteria to cause disease, the method comprising administering to a mucosal surface of the individual a composition comprising a teichoic acid-containing cell polysaccharide (CPS) preparation of Streptococcus pneumoniae in a pharmaceutically acceptable carrier.

2. The method of claim 1, wherein the disease is pneumonia.

3. The method of claim 1, wherein the disease is otitis media.

4. The method of claim 1, wherein the disease is meningitis.

5. The method of claim 1, wherein the disease is bacteremia.

6. A method for inducing an immune response against S. pneumoniae in an individual, the method comprising administering to a mucosal surface of the individual a composition comprising a teichoic acid-containing cell polysaccharide (CPS) preparation of Streptococcus pneumoniae in a pharmaceutically acceptable carrier, wherein the CPS elicits an immune response in the individual.

7. The method of claim 6, wherein the immune response is CD4⁺ T cell mediated.

8. The method of claim 1, 2, 3, 4, 5 or 6 wherein the mucosal surface is associated with nasopharyngeal, oropharyngeal or pharyngeal surface.

9. A method for inhibiting S. pneumoniae bacterial colonization and reducing the ability of the Streptococcus pneumoniae bacteria to cause disease, the method comprising administering through an oral, enteral or pulmonary route a composition comprising a teichoic acid-containing cell polysaccharide (CPS) preparation of Streptococcus pneumoniae in a pharmaceutically acceptable carrier to a mucosal surface of the individual.

10. The method of claim 1 or 6 wherein the teichoic acid-containing CPS preparation further comprises an adjuvant.

11. The method of claim 1 or 6 wherein the teichoic acid-containing CPS preparation comprises a repeating unit of: [chemical structure]
